(19) United States
(12) Patent Application Publication
(10) Pub. No.: US 2015/0209522 A1
(43) Pub. Date: Jul. 30, 2015

(54) DRUG DELIVERY DEVICE AND METHOD FOR DETECTING CONTACT BETWEEN PISTON ROD AND CARTRIDGE BUNG VIA VIBRATION EXCITATION AND MONITORING

(71) Applicant: SANOFI-AVENTIS DEUTSCHLAND GMBH, Frankfurt am Main (DE)

(72) Inventors: Michael Jugl, Frankfurt am Main (DE); Axel Teucher, Frankfurt am Main (DE)

(21) Appl. No.: 14/421,018

(22) PCT Filed: Aug. 19, 2013
(86) PCT No.: PCT/EP2013/067222

§ 371 Date: Feb. 11, 2015

(30) Foreign Application Priority Data
Aug. 20, 2012 (EP) 12180961.0

(51) Int. Cl. A61M 5/315; G01H 1/00

(52) U.S. Cl. CPC A61M 5/31515; G01H 1/00; A61M 2205/3327; A61M 2205/3375; A61M 2207/00

(57) ABSTRACT

The present invention is directed to a method for detecting a contact between a drive mechanism and a bung in a drug delivery device. The bung is movably provided in a cartridge and the drive mechanism includes a piston rod and a bearing for driving the bung in a distal direction. The contact is indicated by a change in a vibration behavior, wherein the method includes the steps of inducing vibration excitations into the drug delivery device, displacing the piston rod relative to the bung such that a gap between the bearing and the bung is reduced and monitoring vibration of the drug delivery device. The invention is further directed to drug delivery device produced according to the respective method.

### CROSS REFERENCE TO RELATED APPLICATIONS

The present application is a U.S. National Phase Application pursuant to 35 U.S.C. §371 of International Application No. PCT/EP2013/067222 filed Aug. 19, 2013, which claims priority to European Patent Application No. 12180961.0 filed Aug. 20, 2012. The entire disclosure contents of these applications are herewith incorporated by reference into the present application.

### TECHNICAL FIELD

The present invention is directed at a method for detecting a contact between a drive mechanism and a bung in a drug delivery device, said bung being movably provided in a cartridge and said drive mechanism including a piston rod and a bearing for driving the bung in a distal direction for delivering a medicament such as insulin. The invention is further directed at a drug delivery device produced by said method.

### BACKGROUND

Pen type drug delivery devices have applications where regular injection by persons without formal medical training occurs. This is increasingly common among patients having diabetes or the like. Self-treatment enables such patients to conduct effective management of their disease. The injection pens usually comprise a housing in which the drive mechanism is located. Some kinds of drug delivery devices also comprise a compartment to accommodate a cartridge in which the medicament is received. With the drive mechanism, the bung in the cartridge is displaced for dispensing the medicament accommodated therein. The drive mechanism includes a piston rod that has a bearing at one end, wherein the bearing is arranged in such manner such that it faces the bung. With the piston rod, the bearing is displaced toward the bung and urges the bung toward a distal end of the drug delivery device, which is closest to the dispensing end (needle end) of the device. Medicament from the cartridge is dispensed thereby. The opposite side of the device is referred to as the proximal end.

In devices of the generic kind, the manufacture may bring unavoidable tolerances and functional clearances between the single components of the drug delivery device, in particular the drive mechanism. As a consequence, clearances such as a gap between the elements of the drive mechanism, such as between the bearing and the cartridge bung may occur even after the drug delivery device has been assembled so that the bung may not be in contact with the distal end of the bearing. It is, therefore, important to eliminate the gap between the cartridge bung and the distal end of the bearing and to bring the drive mechanism in a prestressed state prior to use. Otherwise, it may be possible that the dialed dose may not be dispensed from the device correctly. Initial clearances may already falsify the setting of the dose. To adjust the drug delivery device for use, priming actions are conducted to ensure that the drive mechanism is correctly adjusted, e.g. that the drive mechanism is in contact with the bung so that the correct amount of the medicament can expelled from the device. These actions often come along with a small amount of medicament being dispensed which gives a visual indication that the drug delivery device is ready to use.

It is known in the art to conduct adjustment of the drug delivery device by measurement of the bearing and the bung position before pressing, resp. assembly. The parts are then adjusted according to the measured value such that the bearing is brought into contact with the bung. However, the assembly machines for this method are expensive and the required time cycle is very long.

### SUMMARY

It is an object of the present invention to simplify the adjustment process in a drug delivery device. This object is solved by a method as defined by claim 1 and by a drug delivery device as defined by claim 6.

The present invention is based on the idea that it is possible to detect the contact between the bearing and the cartridge bung in a drug delivery device, such as a an injection pen, during assembly based on a change in the vibration behavior of the device, e.g. a change of the eigenfrequency, after the contact of the bearing and the bung compared with the behavior prior to this contact. The inventive method includes the steps of inducing vibration excitations into the drug delivery device, displacing the piston rod in direction of the bearing and monitoring the resulting vibration of the drug delivery device.

The initial oscillation that is given on the drug delivery device before the bearing contacts the bung, is transferred through its components that are mechanically connected or coupled to each other. By applying a vibration onto the drug delivery device, the vibration system that is constituted by the sum and by the arrangement of the device's components, is encouraged to oscillate. This oscillation can be monitored and detected with suitable means. At least parts of the drug delivery device will oscillate in a certain manner, e.g. at a certain frequency or with characteristic oscillation amplitudes.

When the piston rod, which is displaced toward the bung and the bearing contacts the bung, these elements are then mechanically coupled. In terms of vibrations the mechanical connection results in new oscillation properties that influence the oscillation feedback of the drug delivery device and will change the monitored oscillation characteristics. This can be a change in frequency or in the amplitudes of the oscillation. By monitoring the vibration feedback from the drug delivery device, the moment, the drive mechanism is connected to the bung is indicated by a change in the vibration or oscillation feedback signal. With the measures provided by the invention, an effective way to eliminate further priming actions is offered. The proposed method requires no further elements to be conducted as the method is based on the use of the device's components.

Detecting the contact between the bearing and the bung can be used as a trigger to stop further displacement of the piston rod so that the drug delivery device is optimally prepared. By these measures, the drug delivery device may be prepared in an optimal prestressed condition right after manufacturing the further priming actions are indispensable.

For measuring the vibration feedback, different ways to detect the oscillation feedback are suitable. Accordingly, piezoelectric sensors, laser vibrometers, accelerometers or any other suitable oscillation detecting means are possible. It is, e.g. also possible to detect the audible feedback by a microphone or the like.

It is possible to excite vibration of any component of the device. According to a further embodiment of the invention, at least the drive mechanism is vibrated.

It is also possible to direct the excite vibration at least to the housing. This offers a very convenient way to detect the contact between the drive mechanism and the bung, as the oscillations can be induced into the drug delivery device from the outside, reducing the time for preparing the device.

Preferably, the vibration of the drug delivery device is measured at the same component of the drug delivery device into which the vibration is induced. By these measures, only a small section of the device is necessary for preparation.

Preferably, the piston rod is displaced in distal direction along a helical path with a rotary component and a translational component. The piston rod may be a lead screw which can be in threaded engagement with a body. Advantageously, the body is at least partly surrounding the lead screw. The position of the piston rod relative to the bung can be adjusted by applying torque to the lead screw or to the body.

The object of the invention is further achieved by a drug delivery device which is produced to any of the methods described herein. Such device is well prepared and reuses possible risks to the user.

It is preferred when the cartridge of the drug delivery device is filled with a medicament.

Also, the drug delivery device can be a disposable ejection device. Such devices can be thrown away or recycled after the content of the medicament has been exhausted. However, the present invention is also applicable with re-usable devices designed to replace an emptied cartridge with a filled one and to the content with the former cartridge has been administered.

An example of a disposable device in which the present invention may be used is given in EP 1 974 761 A2.

The term “medicament”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyhepta [symbol] decanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:
H-(Lys)4-des Pro36, des Pro37 Exendin-4(1-39)-NH2,
H-(Lys)5-des Pro36, des Pro37 Exendin-4(1-39)-NH2,
des Pro36 Exendin-4(1-39),
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39); or
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39),
wherein the group -Lys6-NH2 may be bound to the C-terminus of the Exendin-4 derivative;
or an Exendin-4 derivative of the sequence
des Pro36 Exendin-4(1-39)-Lys6-NH2 (AVE0010),
H-(Lys)6-des Pro36 [Asp28] Exendin-4(1-39)-Lys6-NH2,
des Asp28 Pro36, Pro37, Pro38Exendin-4(1-39)-NH2,
H-(Lys)6-des Pro36, Pro38 [Asp28] Exendin-4(1-39)-NH2,
H-Asn-(Glu)5des Pro36, Pro37, Pro38 [Asp28] Exendin-4(1-39)-NH2,
des Pro36, Pro37, Pro38 [Asp28] Exendin-4(1-39)-(Lys)6-NH2,
H-(Lys)6-des Pro36, Pro37, Pro38 [Asp28] Exendin-4(1-39)-(Lys)6-NH2,
H-Asn-(Glu)5-des Pro36, Pro37, Pro38 [Asp28] Exendin-4(1-39)-(Lys)6-NH2,
H-(Lys)6-des Pro36 [Trp(O2)25, Asp28] Exendin-4(1-39)-Lys6-NH2,
H-des Asp28 Pro36, Pro37, Pro38 [Trp(O2)25] Exendin-4(1-39)-NH2,
H-(Lys)6-des Pro36, Pro37, Pro38 [Trp(O2)25, Asp28] Exendin-4(1-39)-NH2,
H-Asn-(Glu)5-des Pro36, Pro37, Pro38 [Trp(O2)25, Asp28] Exendin-4(1-39)-NH2,
des Pro36, Pro37, Pro38 [Trp(O2)25, Asp28] Exendin-4(1-39)-(Lys)6-NH2,
H-(Lys)6-des Pro36, Pro37, Pro38 [Trp(O2)25, Asp28] Exendin-4(1-39)-(Lys)6-NH2,
H-Asn-(Glu)5-des Pro36, Pro37, Pro38 [Trp(O2)25, Asp28] Exendin-4(1-39)-(Lys)6-NH2,
H-(Lys)6-des Pro36 [Met(O)14, Asp28] Exendin-4(1-39)-Lys6-NH2,
des Met(O)14 Asp28 Pro36, Pro37, Pro38 Exendin-4(1-39)-NH2,
H-(Lys)6-desPro36, Pro37, Pro38 [Met(O)14, Asp28] Exendin-4(1-39)-NH2,
H-Asn-(Glu)5-des Pro36, Pro37, Pro38 [Met(O)14, Asp28] Exendin-4(1-39)-NH2,
des Pro36, Pro37, Pro38 [Met(O)14, Asp28] Exendin-4(1-39)-(Lys)6-NH2,
H-(Lys)6-des Pro36, Pro37, Pro38 [Met(O)14, Asp28] Exendin-4(1-39)-(Lys)6-NH2,
H-Asn-(Glu)5 des Pro36, Pro37, Pro38 [Met(O)14, Asp28] Exendin-4(1-39)-(Lys)6-NH2,
H-Lys6-des Pro36 [Met(O)14, Trp(O2)25, Asp28] Exendin-4(1-39)-Lys6-NH2,
H-des Asp28 Pro36, Pro37, Pro38 [Met(O)14, Trp(O2)25] Exendin-4(1-39)-NH2,
H-(Lys)6-des Pro36, Pro37, Pro38 [Met(O)14, Asp28] Exendin-4(1-39)-NH2,
H-Asn-(Glu)5-des Pro36, Pro37, Pro38 [Met(O)14, Trp(O2)25, Asp28] Exendin-4(1-39)-NH2,
des Pro36, Pro37, Pro38 [Met(O)14, Trp(O2)25, Asp28] Exendin-4(1-39)-(Lys)6-NH2,
H-(Lys)6-des Pro36, Pro37, Pro38 [Met(O)14, Trp(O2)25, Asp28] Exendin-4(S1-39)-(Lys)6-NH2,
H-Asn-(Glu)5-des Pro36, Pro37, Pro38 [Met(O)14, Trp(O2)25, Asp28] Exendin-4(1-39)-(Lys)6-NH2;
   or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (CH) and the variable region (VH). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

In the following, the invention will be described by way of an example and with reference to the schematic drawing which shows a partial sectional view of a drug delivery device in a state before the bearing is in contact with the bung.

### BRIEF DESCRIPTION OF THE FIGURES

FIG. 1 is a partial cross-sectional view of one embodiment of the invention.

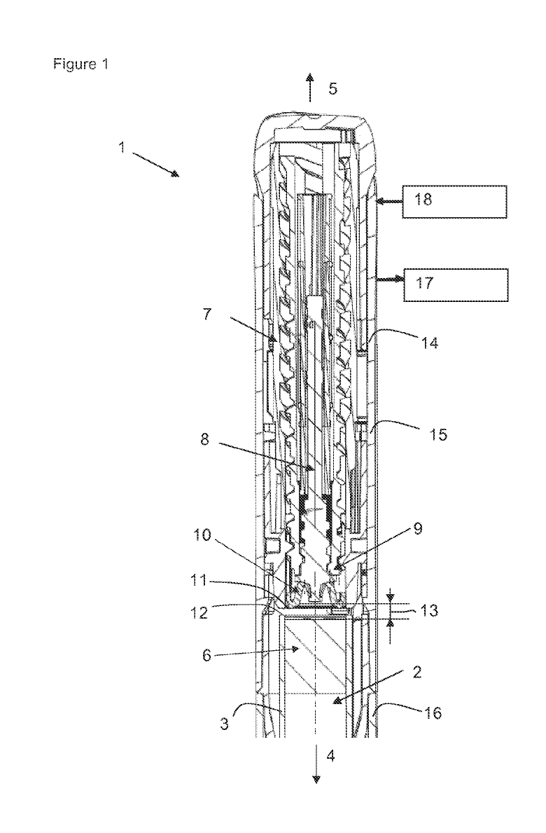

### DETAILED DESCRIPTION

In the figure, there is shown a part of a drug delivery device 1 for expelling a medicament 2 out of a cartridge 3 in a distal direction 4. Opposite the distal end of the drug delivery device 1, in a proximal direction 5, a movable bung 6 is placed in the cartridge 3. A distal movement of the cartridge bung 6 is induced by a drive mechanism 7 located in proximal direction 5 from the cartridge bung 6.

The drive mechanism 7 comprises a piston rod 8, said piston 8 being threadedly engaged with a body 9 that is partially surrounding the piston rod 8. By rotational movement of the piston 8 relative to the body 9, the piston rod 8 can be translated in distal direction. The distal end of the piston rod 8 is provided with a bearing 10 wherein the bearing 10 has a distal end surface 11.

Initially, the bearing 10 and the bung 6 are arranged spaced from each other during assembly of the device, wherein between the distal end surface 11 of the bearing 10 and a proximal surface 12 of the bung 6, a clearing, respectively a gap 13 is present. The components described above are in the shown embodiment arranged in a housing 14, said housing comprising a first part 15 and a second part 16, with the drive mechanism 7 accommodated in the first part 15.

In order to adjust the device 1 such as to eliminate the gap 13 so later priming actions are no longer necessary, the distal end surface 11 of the bearing 10 will be brought into contact with the proximal surface 12 of the bung 6. For this purpose, the drive mechanism can be actuated, whereby the piston rod 8 with the bearing 10 at its distal end is moved in distal direction 4.

During the approaching of the bearing 10, the drug delivery device 1 is excited to vibrate. For this purpose, vibrations, respectively oscillations are induced into the drug delivery device 1 by vibration generating means (vibrator) 17. In the shown embodiment, the oscillation excitations are directly applied to the outside of the first part 15 of the housing 14. The housing 14 is connected to the drive mechanism 7 in such a way that vibrations from the housing 14 are transmitted into the drive mechanism 7 which is respectively excited. Some of the components constituted in the oscillation system that oscillates with specific characteristic are an answer to the induced vibrations from the vibrator 17. This oscillation feedback, respectively oscillation answer is measured by measuring means (oscilloscope) 18.

By means of the drive mechanism 7, the piston rod 8 and the bearing 10 are now moved in distal direction, wherein the gap 13 is gradually reduced until the distal end surface 11 of the bearing 10 contacts the proximal surface 12 of the bung 6. The coupling of the bearing 10 to the bug 6 changes the oscillation characteristic respectively the frequency and/or the amplitude of the oscillation feedback which can be measured with the oscilloscope 18. The change in the feedback signal thereby indicates that the bearing 10 is in contact with the bung 6.

After the connection is established, the adjustment process is finished. Further displacement of the piston rod 8 can be stopped as the monitored feedback signals have indicated that the drug delivery device is now in prestressed and prepared condition.

1-11. (canceled)

12. A method for detecting a contact between a drive mechanism and a bung in a drug delivery device, said bung being movably provided in a cartridge and said drive mechanism including a piston rod and a bearing for driving the bung in a distal direction, wherein the contact is indicated by a change in a vibration behavior, said method including the steps of:
inducing vibration excitations into the drug delivery device;
displacing the piston rod relative to the bung such that a gap between the bearing and the bung is reduced;
monitoring vibration of the drug delivery device.

13. The method according to claim 12, characterized in that further movement of the piston rod in distal direction is stopped when the monitored vibration behavior changes, thus indicating contact between the drive mechanism and the bung.

14. The method according to claim 12, characterized in that at least the drive mechanism is directly excited to vibrate.

15. The method according to claim 12, characterized in that at least a housing of the drug delivery device is directly excited to vibrate.

16. The method according to claim 12, characterized in that the vibration of the drug delivery device is measured at same component of the drug delivery device the vibration is induced.

17. The method according to claim 12, characterized in monitoring the vibration amplitude.

18. The method according to claim 12, characterized in monitoring the vibration frequency.

19. A drug delivery device produced according to claim 12.

20. The drug delivery device according to claim 19, characterized in that the cartridge is filled with a medicament.

21. The drug delivery device according to claim 19, characterized in that the drug delivery device is a disposable injection device.

22. A method for assembling a drug delivery device using the method of claim 12 to detect contact between the drive mechanism and the bung.
